(19) United States
(12) Patent Application Publication
(10) Pub. No.: US 2009/0280110 A1
(43) Pub. Date: Nov. 12, 2009

(54) Cell Model for Alzheimer's Disease Pathology

(75) Inventors: George S. Bloom, Charlottesville, VA (US); Michelle E. King, Ruckersville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/304,805

(22) PCT Filed: Jun. 27, 2007
(86) PCT No.: PCT/US07/14941

§ 371 Date: Dec. 15, 2008

Related U.S. Application Data
(60) Provisional application No. 60/816,717, filed on Jun. 27, 2006.

(51) Int. Cl. A61K 39/395; C12Q 1/02; C40B 30/06; C12Q 1/68; C07H 21/00; C07K 14/00; C07K 16/00; C07H 21/02; A61K 31/7088; A61K 38/00

(52) U.S. Cl. 424/130.1; 435/29; 506/10; 435/6; 536/23.1; 530/350; 530/300; 530/387.1; 536/24.5; 514/44 A; 514/44 R; 514/2

(57) ABSTRACT

The present invention encompasses compositions and methods for studying in cell culture the pathologic changes associated with Alzheimer's disease. The present invention further relates to methods for studying and detecting early events in the conversion of healthy neurons to Alzheimer's disease neurons.

### CROSS REFERENCE TO RELATED APPLICATIONS

This application is entitled to priority pursuant to 35 U.S.C. § 119(e) to U.S. provisional patent application No. 60/816,717, filed Jun. 27, 2006, the entirety of which is incorporated by reference herein.

### STATEMENT REGARDING FEDERALLY SPONSORED RESEARCH AND DEVELOPMENT

This invention was made with United States Government support under Grant Nos. AG20465, AG02665, and NS051764 awarded by the National Institutes of Health. The United States Government may have certain rights in the invention.

### BACKGROUND

Alzheimer's disease (AD) is the leading cause of dementia in the elderly, and is becoming more prevalent as the average human lifespan rises and human populations expand. The symptoms of AD, i.e., reduction of memory and cognitive skills, are caused by the loss or impaired function of synaptic connections between neurons in the brain. At the histopathological level, AD is defined by two types of abnormal structures that accumulate in brain, amyloid deposits and neurofibrillary tangles. Amyloid deposits are extracellular fibrils formed from Aβ40 and Aβ42, peptides that are generated by proteolytic cleavage of β-amyloid precursor protein (APP). Neurofibrillary tangles, in contrast, are intracellular filaments, and are formed by polymerization of tau, which normally serves as a microtubule-associated protein in nerve cell axons.

Much debate has been sparked by the question of whether Aβ fibrils and tau filaments are intrinsically toxic or are simply indicators of more fundamentally harmful neurodegenerative processes. Nevertheless, four sets of observations point to the central involvement of both Aβ and tau in neurodegeneration: 1) AD can be caused by APP mutations that increase the overall level of Aβ or the ratio of Aβ42 to Aβ40 in brain; 2) Tau mutations cause similar neurodegenerative diseases, the non-Alzheimer's tauopathies; 3) Cognitive impairment and extensive neurodegeneration do not occur when amyloid pathology is not accompanied by tau pathology; and 4) Tau pathology in the absence of amyloid pathology is invariably associated with neurodegeneration, as in the non-Alzheimer's tauopathies.

Tau promotes the polymerization of tubulin into microtubules and stabilizes the latter. Binding to microtubules and microtubule assembly requires the “repeat domain” in the C-terminal half of Tau, as well as the two regions flanking the repeats.

Thus, in both AD and non-AD tauopathies, neurodegeneration requires malfunctioning of tau. In the specific case of AD, pathological tau behavior seems to be caused by a process that is initiated upstream by Aβ.

Insoluble deposits of Aβ, in the form of senile plaques, and of tau, as neurofibrillary tangles, have long been accepted as the primary histopathological markers of AD. While initial research focused on the role of Aβ and tau individually, recent evidence, including data demonstrating that amyloid pathology can upregulate tau pathology (Gotz et al., 2001; Lewis et al., 2001), places both Aβ and tau in a signaling cascade pathway that leads from Aβ through tau (Hardy and Selkoe, 2002; Lee et al., 2001; Selkoe, 2001). Regrettably, the key steps within this cascade remain poorly understood.

An exciting new focus of investigation has been on the role that non-fibrillar forms of Aβ, and to a lesser extent tau, play in AD. Soluble forms of Aβ are more potent than fibrillar forms at eliciting cellular responses, such as increased apoptosis (Sponne et al., 2003) and decreased synaptic plasticity (Walsh et al., 2002). In fact, studies of transgenic animal models and AD patients have shown that cognitive deficits and synaptic loss correlate with soluble Aβ, rather than senile plaques (Kayed et al., 2003; Oddo et al., 2005), suggesting that AD is initiated well before extracellular Aβ deposits are evident.

Although the steps connecting Aβ to tau in AD remain very poorly understood, it follows naturally that a better understanding of how tau establishes the toxicity of Aβ might lead directly to effective clinical management of AD.

There is a long felt need in the art for compositions and methods to study, treat, and diagnose Alzheimer's disease. The present invention satisfies these needs.

### SUMMARY OF INVENTION

The present application discloses cell culture models that detect the earliest known cell biological event in conversion of healthy neurons to AD neurons.

The present invention provides methods using cultured neuronal and non-neuronal cells to model tau-dependent effects on microtubules of various forms of Aβ. Microtubules are required for maintenance of synaptic connections between both pre-synaptic and post-synaptic neurons. To model how Aβ signals to tau, tau was expressed by transient transfection in cultured fibroblasts, which do not express their endogenous tau gene, and then various forms of Aβ, including pre-fibrillar and fibrillar Aβ40 and Aβ42 were added to the culture medium. Remarkably, it was found that brief exposure of cells to submicromolar levels of pre-fibrillar Aβ42 caused massive and rapid, tau-dependent disassembly of microtubules. Similar results were obtained for pre-fibrillar Aβ40, albeit at much higher concentrations, but microtubules in either tau-expressing or tau-deficient cells were resistant to fibrillar Aβ. Related experiments in primary cultured neurons, which express tau endogenously, confirmed the results obtained in cultured fibroblasts.

Taken together, these results highlight the most dramatic, rapid and sensitive link between Aβ and tau described to date, identify microtubules as primary, tau-dependent targets of Aβ, and suggest that non-fibrillar Aβ and tau underlie the synaptic loss and detrimental neurodegeneration observed in AD prior to the accumulation of fibrillar forms in senile plaques and neurofibrillary tangles.

In one embodiment, the present invention provides a method of screening for compounds which inhibit the signaling pathway between Aβ and tau and microtubule disassembly. In one aspect, compounds identified by the methods of the invention are useful to stimulate pre-fibrillar Aβ to form fibrils. In one aspect, the compounds of the invention prevent or inhibit tau dissociation from microtubules or a change in tau's interaction with microtubules. In another aspect, compounds of the invention are useful for preventing or inhibiting microtubule disassembly. The present invention further encompasses compounds identified by the methods of the invention. In one aspect, compounds identified by the methods of the invention are useful for treating diseases. In one aspect, the disease is Alzheimer's disease.

Moreover, the experimental approach disclosed herein is readily adaptable to various techniques for identifying compounds with the desired biological activity. In one aspect, the methods of the invention are useful for high throughput screens for revealing the biochemical connections between Aβ and tau, and for finding compounds that block the connection, and by extension, may block progression of AD. In one aspect, AD is prevented, or its progression inhibited, by inhibiting the signaling pathway from Aβ to tau and microtubule disassembly. This includes inhibiting upstream regulatory pathways of Aβ which are associated with the initiation or progression of AD via the Aβ-tau pathway.

In one aspect, the invention encompasses the use of high throughput screening of siRNA and combinatorial chemical libraries. For example, cells that express fluorescent tau and tubulin can be plated in multi-well dishes (24-96 wells, for example), each well of which is treated with a specific, known siRNA or compound generated by combinatorial chemistry. Cells can then be exposed to pre-fibrillar Aβ42, and after a suitable incubation period, subjected to fluorescence microscopy. The use of robotic instruments to manipulate the cell cultures, and an automated fluorescence microscope that can be programmed to photograph cells in each well will enable high throughput screening to be accomplished. Of course, human inspection of the photographs will be needed to judge the results of such screens. If a particular siRNA protects microtubules from Aβ in tau-expressing cells, it is likely that the protein knocked down by that siRNA is a requisite intermediate for pathological signaling from Aβ to tau. Likewise, any combinatorially generated compound with a similarly protective effect would be a candidate therapeutic agent for AD. The timing and order in which the cells are contacted with test compounds can vary. In one aspect, Aβ can be Aβ40 and/or Aβ42. In one aspect, Aβ can be fibrillar and/or pre-fibrillar.

The invention further encompasses biochemical assays for microtubules that are useful for high throughput, robotically controlled screens. For example, see FIGS. 1D, 2C, and 2D. Other biochemical assays are known in the art as well.

It is disclosed herein that that tau confers acute hypersensitivity of microtubules to pre-fibrillar, extracellular Aβ. Therefore, it will be understood by one of ordinary skill in the art that the present invention encompasses methods to identify compounds which decrease this sensitivity. The present invention further provides for the use of such compounds.

It is also disclosed herein that the active region of tau is localized to an N-terminal domain that does not bind microtubules and is not part of the region of tau that assembles into filaments, but does respond to pre-fibrillar Aβ42. Therefore, the present invention encompasses methods of identifying inhibitors of the interaction of the N-terminal domain of tau with pre-fibrillar Aβ42 to prevent or inhibit the initiation or progression of AD and similar events. These results suggest that a seminal cell biological event in AD pathogenesis is acute, tau-dependent loss of microtubule integrity caused by exposure of neurons to readily diffusible Aβ.

It will be appreciated by those of ordinary skill in the art that the model described herein is useful for defining the biochemical steps that underlie tau-dependent microtubule poisoning by pre-fibrillar Aβ. In one aspect, the methods of the invention are useful to identify compounds that block or interfere with pathogenic signaling from pre-fibrillar Aβ to tau. The model described herein is also useful for identifying compounds which directly modulate the interaction of tau with microtubules. It will be appreciated by one of ordinary skill in the art that when a test compound is used, the timing of when it is added, versus when Aβ is added, will help determine which pathway or interaction upon which the compound is acting.

It will be further appreciated that the present model is useful for analyzing effects of tau on microtubules which are independent of Aβ effects on tau or microtubules. The invention thus encompasses adding test compounds without adding Aβ. The invention further encompasses testing tau by comparing cells with and without tau.

In one embodiment, the present invention encompasses a method of identifying a compound that inhibits Aβ or tau mediated disassembly of microtubules. The method comprises culturing cells comprising tau and tubulin, contacting said cells with at least one test compound, optionally contacting said cells with Aβ, and analyzing the microtubules in the treated cells and comparing the microtubules to those in otherwise identical cells not contacted with a test compound. Finding a higher level of microtubules in the treated cells is an indication that the test compound inhibits Aβ or tau mediated disassembly of tau with microtubules. In one aspect, the invention encompasses identifying a compound wherein said compound inhibits the interaction of the N-terminal domain of tau with said Aβ. In another aspect, the method of the invention is useful for identifying inhibitors of the interaction of the N-terminal domain of tau with said Aβ. The method can be practiced using Aβ or pre-fibrillar A\tilde{β}

In one aspect, the methods of the present invention are useful for identifying compounds that regulate the interaction of tau with tubulin. In another aspect, the method is useful when a change in the interaction of tau with tubulin, or interaction of Aβ with tau, is associated with microtubule disassembly. In yet another aspect, the method of the present invention is useful for identifying compounds that reduce the sensitivity of microtubule disassembly to tau mediated by Aβ. In a further aspect, the method is useful for identifying compounds that inhibit the interaction of tau with Aβ. One of ordinary skill in the art will appreciate that additional methods and assays are available for further clarifying exactly what the compound inhibits.

In one aspect, the Aβ tested can be fibrillar or prefibrillar. In one aspect, the Aβ can be Aβ40 or Aβ42

In one embodiment, the cells used in the practice of the invention are primary cells. In one aspect, the primary cells are non-neuronal cells. In one aspect, the primary cells are primary neuronal cells. In one aspect, the primary neuronal cells are primary hippocampal cells.

In one embodiment, the cells used in the practice of the invention are non-primary cells. In one aspect, the non-primary cells are a non-primary cell strain. It will be appreciated that the cells can be a cell line of clonal origin or cells of non-clonal origin. In one aspect, the cells of the invention are neuronal. In another aspect, the cells are non-neuronal. In one aspect, the non-neuronal cells are fibroblasts.

In one embodiment, tau is not endogenously expressed in the cells and is transfected into the cells. In one aspect, the proteins can be labeled with detectable markers to allow detection, analysis, and visualization of the proteins. In one aspect, the label is a fluorescent marker. One of ordinary skill in the art will appreciate that the cultures can be set up to encompass high throughput screening techniques to identify compounds of interest. In one aspect, the compound is a chemical. In one aspect, the chemical is identified as part of a combinatorial library. In yet another aspect, the compounds of the invention include, but are not limited to, compounds such as interfering RNA, a small interfering RNA, an oligonucleotide, a protein, a peptide, an antibody, and an aptamer.

The present invention further encompasses compounds identified by the methods described herein.

In one aspect, Aβ is added to the culture before a test compound is added. In another aspect, Aβ is added to the culture after a test compound is added to the culture.

In one embodiment, the present invention encompasses the compounds identified by the methods of the invention. The present invention further provides pharmaceutical compositions comprising compounds identified by the methods of the invention.

In one embodiment, the present invention encompasses an in vitro model for detecting and measuring early cellular events in Alzheimer's disease. The model comprises culturing cells in vitro, wherein the cells comprise tau and tubulin, contacting the cells with Aβ, and analyzing changes in microtubules. As described above, this model is also useful for identifying compounds which regulate processes associated with Alzheimer's disease. In one aspect of the in vitro model, the cells do not endogenously express tau. In one aspect, the cells are CV-1 African green monkey kidney cells. In one aspect, the cells are human cells.

In one embodiment, a compound identified by the method of the invention is useful for treating Alzheimer's disease. In one aspect, the invention provides methods for administering a compound identified by the method of the invention to a subject in need thereof. Optionally, an Alzheimer's disease therapeutic agent can be administered with a compound of the invention.

Various assays for analyzing tau and Aβ interactions and microtubule disassembly are described herein or are known in the art, and are encompassed within the present invention.

### BRIEF DESCRIPTION OF THE DRAWINGS

FIG. 1, comprising FIGS. 1a, 1b, and 1c, represent photomicrographs of an experiment demonstrating Tau-dependent hypersensitivity of CV-1 cell microtubules to pre-fibrillar Aβ42. CV-1 cells transfected with tau-CFP and YFP-tubulin were treated with pre-fibrillar Aβ42 as indicated, and imaged by time lapse fluorescence microscopy. FIG. 1a comprises images of eight micrographs, taken at 0, 20, 60, and 120 minutes post-treatment (upper panel—tau; lower panel—tubulin). 1 μM pre-fibrillar Aβ42 caused tau to dissociate from microtubules, and the microtubules to disassemble soon thereafter. FIG. 1b comprises four photomicrographs depicting tubulin at 0, 30, 60, and 140 minutes post-treatment in cells not expressing tau. This effect required tau expression, because microtubules remained intact in cells that expressed only YFP-tubulin and were treated with 3 μM pre-fibrillar Aβ42. FIG. 1c comprises four photomicrographs depicting tubulin in tau-expressing cells at 0, 30, 60 and 140 minutes post-treatment. Microtubules were unaffected in tau-expressing cells exposed to 3 μM fibrillar Aβ42. FIGS. 1d and 1e demonstrate time-dependent microtubule loss induced by prefibrillar Aβ42 documented in 1d by fractionation of tubulin into soluble (S) and polymerized (P) pools (Black et al., 1996), and quantitation of fluorescence micrographs of fixed cells expressing Tau-CFP and counterstained with anti-tubulin (1e). FIG. 1d comprises upper (tubulin) and lower (Tau-YFP) panels representing blots at times of 0, 30 minutes, and 2 hours. FIG. 1e is a graphic representation of the quantitative results obtained from the analysis of the prior figures. The ordinate of FIG. 1e represents the percentage of cells with microtubules. The four groups of time in 1e are 0 hr, 0.5 hr, 2.0 hr, and 3.0 hr. Error bars in FIG. 1e indicate the SD, and transfected and nontransfected refer to cells that did and did not express Tau-CFP, respectively.

FIG. 2, comprising FIGS. 2a-2d, represents the results of experiments demonstrating Tau-dependent hypersensitivity of neuronal microtubules to pre-fibrillar Aβ42. Primary rat cortical neurons were cultured for at least 8 days before treatment with pre-fibrillar Aβ42. FIG. 2a represents images of eight photomicrographs. Cells were stained by immunofluorescence for tubulin (DM1α) (upper panel; four images) and tau (R1 tau) (lower panel; four images) at the timepoints indicated following the addition of 1 μM pre-fibrillar Aβ42. Note the swollen varicosities induced by the Aβ42. FIG. 2b represents images of four electron micrographs of cells cultured under similar conditions to those represented by FIG. 2a. Neurites in neurons treated for 2 hours with 1 μM pre-fibrillar Aβ42 were found by electron microscopy to contain numerous varicosities that were filled with membrane-bound organelles and lacked microtubules, and additional regions with sparse, poorly organized microtubules. FIG. 2c represents an image (six lanes) of an assay which partitions un-polymerized and polymerized tubulin. Primary hippocampal neurons (similar results were obtained for cortical neurons) were extracted with Triton X-100 to separate the soluble (S) from polymerized (P) tubulin (Black et al., 1996). Note that ˜90% of the tubulin was polymerized in control cultures (left two lanes), that only ˜45% was polymerized after 2 hours of cellular exposure to 1 μM pre-fibrillar Aβ42 (middle two lanes), and that only a modest loss of polymerized tubulin (65% polymerized) was caused by a 2 hour exposure to 3 μM fibrillar Aβ42 (right two lanes). FIG. 2d represents images (left and right panel) of an assay where primary neurons were transfected with a tau specific siRNA and treated with 2 μM pre-fibrillar Aβ42 for 2 hours prior to extraction. Left Panel: (left lane—control siRNA; right lane—Tau siRNA; upper bands—tubulin; lower bands—tau 5). Right Panel: control (S and P), first two lanes; 2 mM β-amyloid at 2 hours (S and P), right two lanes; upper bands—Tau siRNA; lower bands—control siRNA. The siRNA-treated cells expressed between 1/16 and 1/32 the normal level of tau, and showed no change in tubulin levels in response to the Aβ42 treatment when compared to the tau expressing cells.

FIG. 3 represents images of western blots demonstrating that Pre-fibrillar Aβ42 does not induce AD-like tau phosphorylation. Primary rat hippocampal neurons were treated with 1 μM Aβ42 for 2 hours prior to western blotting with the indicated antibodies: Tau 5 (upper panel), Tau-1 (second panel; (dephospho-serine 199 and dephospho-serine 202)), AT180 (third panel; phospho-serine 231), PHF-1 (fourth panel; phospho-serine 396 and phospho-serine 404), and actin (fifth panel). The lanes, left to right, are-control, 1 μM Aβ42, AD Brain, and purified PHF. The phosphorylation of tau in treated neurons was compared to whole brain extract from an AD brain, as well as to paired helical filaments purified from an AD brain. Note that AD-like tau phosphorylation was not increased by pre-fibrillar Aβ42.

FIG. 4, comprising FIGS. 4a and 4b, represents schematics and photomicrographic images illustrating that the active portion of tau resides within an N-terminal fragment that does not target to microtubules. CV-1 cells transfected with the indicated fluorescently tagged proteins were treated with 1 μM pre-fibrillar Aβ42, and imaged by time lapse fluorescence microscopy. FIG. 2a comprises three schematics of domains of the expression vectors used (MAP2C (top), MAP2C chimera (middle), and Tau Chimera (bottom) on the left side of the figure), and on the right are 12 photomicrographic images (3 rows of four images each; top, middle, and bottom) corresponding to the use of the domains. The cells were treated for 0, 30, 60, or 140 minutes in the upper two groups, and for 0, 20, 30, and 40 minutes in the lower group. Microtubules remained intact in cells expressing GFP-MAP2c or GFP-MAP2c chimera after more than 2 hours of exposure to Aβ42. In contrast, microtubules depolymerized in cells expressing GFP-tau chimera after less than an hour of exposure to Aβ42. FIG. 2b represent schematics (left) of tubulin (upper schematic) and the tau projection domain (lower schematic) and eight photomicrographs (right) demonstrating that microtubules also depolymerized in cells that were exposed to Aβ42 and expressed the N-terminal arm of tau coupled to CFP. The bar in FIGS. 4a and 4b indicates 20 microns.

FIG. 5 graphically illustrates the results of quantitation of fluorescence micrographs for Aβ42-induced microtubule loss. CV-1 cells expressing the indicated proteins with 40-50% transfection efficiency were exposed to the form of Aβ42 specified on the figure. The cells were then fixed and stained with anti-tubulin and scored for microtubules as described in Materials and methods. Transfected and nontransfected refer to cells that did and did not express the indicated transgenes, respectively. Pairwise comparisons were made of transfected versus nontransfected cells at 0 hours and 3 hours of Aβ exposure and of nontransfected cells at 0 versus 3 h of Aβ exposure. Error bars indicate the SD, and asterisks mark statistically significant differences at =0.02 between the indicated pairs of transfected and nontransfected cells after 3 h of Aβ exposure. Statistically significant differences were not found for any pair of transfected versus nontransfected cells at 0 b, nor for any nontransfected pair at 0 versus 3 h. The collective results shown here confirm the qualitative results shown in FIGS. 1 and 4 (Supplemental figures are also available at the Journal of Cell Biology website for King et al., 2006, J. Cell Biol.). The ordinate represents 5 cells with microtubules. The groups are: Tau-YFP pre-fibrillar Aβ42 (1 μM); Tau-YFP fibrillar Aβ42 (3 μM); GFP-MAP2c pre-fibrillar Aβ42 (1 μM); GFP-MAP2c Chimera pre-fibrillar Aβ42 (1 μM); GFP-Tau Chimera pre-fibrillar Aβ42 (1 μM); and Tau Arm-CFP pre-fibrillar Aβ42 (1 μM).

### DETAILED DESCRIPTION OF THE INVENTION

In describing and claiming the invention, the following terminology will be used in accordance with the definitions set forth below.

### DEFINITIONS, ABBREVIATIONS, AND EPONYMS

AD—Alzheimer's Disease
Aβ—β-amyloid
APP—β-amyloid precursor protein
P—polymerized
S—soluble

As used herein, the articles “a” and “an” refer to one or to more than one, i.e., to at least one, of the grammatical object of the article. By way of example, “an element” means one element or more than one element.

By the term “Aβ or tau mediated” is meant regulation by either or both of Aβ and tau, not just one or the other.

A disease, condition, or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, an “analog” of a chemical compound is a compound that, by way of example, resembles another in structure but is not necessarily an isomer (e.g., 5-fluorouracil is an analog of thymine).

As used herein, the term “affected cell” refers to a cell of a subject afflicted with a disease or disorder, which affected cell has an altered phenotype relative to a subject not afflicted with a disease or disorder.

Cells or tissue are “affected” by a disease or disorder if the cells or tissue have an altered phenotype relative to the same cells or tissue in a subject not afflicted with a disease or disorder.

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1988; Houston et al., 1988; Bird et al., 1988).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

The term “cell” as used herein, may be used to encompass the terms “cell strain” and “cell line”. However, the terms “cell strain” and “cell line” are specifically used herein when referring specifically to a “cell strain” or a “cell line”. All of these terms also include their progeny, which are any and all subsequent generations. It is understood that all progeny may not be identical due to deliberate or inadvertent mutations, particularly in cells lines. A culture of cells with an indefinite life span is considered immortal; such a culture is called a “cell line”, to distinguish it from an impermanent “cell strain”. A lineage of cells originating from a primary culture is called a “cell strain”.

The terms “cell culture” and “culture,” as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) may then be used to support the growth or differentiation of a second population of cells.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, but are not limited to, radioactive isotopes, antigenic determinants, enzymes, nucleic acids available for hybridization, chromophores, fluorophores, chemiluminescent molecules, electrochemically detectable molecules, and molecules that provide for altered fluorescence polarization or altered light scattering.

As used herein, a “derivative” of a compound refers to a chemical compound that may be produced from another compound of similar structure in one or more steps, as in replacement of H by an alkyl, acyl, or amino group.

As used herein, an “effective amount” means an amount sufficient to produce a selected or desired effect.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “endogenously express” refers to the situation where a protein or mRNA is normally expressed in at least detectable levels in a cell. By the term “does not endogenously express” is meant a protein or mRNA which is expressed at extremely low or undetectable levels, or not at all.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly 5 amino acids or sugars in size. One skilled in the art understands that generally the overall three-dimensional structure, rather than the specific linear sequence of the molecule, is the main criterion of antigenic specificity.

The term “expression” as used herein is defined as the transcription and/or translation of a particular nucleotide sequence driven by its promoter.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. The terms, “feeder cells”, “feeders,” and “feeder layers” are used interchangeably herein.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

The term “ingredient” refers to any compound, whether of chemical or biological origin, that can be used in cell culture media to maintain or promote the growth or proliferation of cells. The terms “component,” “nutrient” and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit,” as used herein, refers to the ability of a compound of the invention to reduce or impede a described function. Preferably, inhibition is by at least 10%, more preferably by at least 25%, even more preferably by at least 50%, and most preferably, the function is inhibited by at least 75%.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See the National Institutes of Health website.

The percent identity between two sequences can be determined using techniques similar to those described above, with or without allowing gaps. In calculating percent identity, typically exact matches are counted.

The term “inhibit,” as used herein, refers to the ability of a compound or any agent to reduce or impede a described function. Preferably, inhibition is by at least 10%, more preferably by at least 25%, even more preferably by at least 50%, and most preferably, the function is inhibited by at least 75%. The term “inhibit” is used interchangeably with “prevent” and “block”.

The term “inhibit a complex”, as used herein, refers to inhibiting the formation of a complex or interaction of two or more proteins, as well as inhibiting the function or activity of the complex. The term also encompasses disrupting or inhibiting the normal interaction of said proteins. For example, Aβ interaction with tau, which disrupts the normal interaction of tau with microtubules and subsequent disassembly of microtubules, is considered an abnormal interaction, even if tau is still interacting with microtubules, particularly since the present data suggest that tau interaction with microtubules confers hypersensitivity of microtubules to disassembly when Aβ interacts with tau. The term also encompasses disrupting a formed complex. However, the term does not imply that each and every one of these functions must be inhibited at the same time. The terms “inhibit a complex” and “inhibit interaction” are used interchangeably herein.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

The term “level of microtubules”, as used herein, refers to the amount of microtubules. Such an amount can be visualized microscopically as described herein, and can be determined using biochemically as well.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

The term “modulate”, as used herein, refers to changing the level of an activity, function, or process. The term “modulate” encompasses both inhibiting and stimulating an activity, function, or process.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “nucleic acid construct,” as used herein, encompasses DNA and RNA sequences encoding the particular gene or gene fragment desired, whether obtained by genomic or synthetic methods.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

The term “protein” typically refers to large polypeptides.

The term “peptide” typically refers to short polypeptides.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

“Plurality” means at least two.

As used herein, the term “purified” and like terms relate to an enrichment of a cell, cell type, molecule, or compound relative to other components normally associated with the cell, cell type, molecule, or compound in a native environment. The term “purified” does not necessarily indicate that complete purity of the particular cell, cell type, molecule, or compound has been achieved during the process.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

The term “regulate” refers to either stimulating or inhibiting a function or activity of interest. For example, identifying a compound which “regulates the interaction of tau with microtubules” in the context of the present invention would mean that the “regulation” results in a change in the interaction leading to less disassembly of microtubules.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

The term “standard,” as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In one aspect, the activity or differentiation is stimulated by at least 10% compared to a control value, more preferably by at least 25%, and even more preferably by at least 50%. The term “stimulator” as used herein, refers to any compound or agent, the application of which results in the stimulation of a process or function of interest, including, but not limited to, osteoclast production, differentiation, and activity.

A “subject” of analysis, diagnosis, or treatment is an animal. Such animals include mammals, preferably a human.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Chemical Definitions

As used herein, the term “halogen” or “halo” includes bromo, chloro, fluoro, and iodo.

The term “haloalkyl” as used herein refers to an alkyl radical bearing at least one halogen substituent, for example, chloromethyl, fluoroethyl or trifluoromethyl and the like.

The term “C₁-Cₙ alkyl” wherein n is an integer, as used herein, represents a branched or linear alkyl group having from one to the specified number of carbon atoms. Typically, C₁-C₆ alkyl groups include, but are not limited to, methyl, ethyl, n-propyl, iso-propyl, butyl, iso-butyl, sec-butyl, tert-butyl, pentyl, hexyl, and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n=8, represents cyclopropyl, cyclobutyl, cyclopentyl, cyclohexyl, cycloheptyl, and cyclooctyl.

As used herein, the term “optionally substituted” refers to from zero to four substituents, wherein the substituents are each independently selected. Each of the independently selected substituents may be the same or different than other substituents.

As used herein the term “aryl” refers to an optionally substituted mono- or bicyclic carbocyclic ring system having one or two aromatic rings including, but not limited to, phenyl, benzyl, naphthyl, tetrahydronaphthyl, indanyl, indenyl, and the like.

“Optionally substituted aryl” includes aryl compounds having from zero to four substituents, and “substituted aryl” includes aryl compounds having one or more substituents. The term (C₅-C₈ alkyl)aryl refers to any aryl group which is attached to the parent moiety via the alkyl group.

The term “heterocyclic group” refers to an optionally substituted mono- or bicyclic carbocyclic ring system containing from one to three heteroatoms wherein the heteroatoms are selected from the group consisting of oxygen, sulfur, and nitrogen.

As used herein the term “heteroaryl” refers to an optionally substituted mono- or bicyclic carbocyclic ring system having one or two aromatic rings containing from one to three heteroatoms and includes, but is not limited to, furyl, thienyl, pyridyl and the like.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The compounds of the present invention contain one or more asymmetric centers in the molecule. In accordance with the present invention a structure that does not designate the stereochemistry is to be understood as embracing all the various optical isomers, as well as racemic mixtures thereof.

The compounds of the present invention may exist in tautomeric forms and the invention includes both mixtures and separate individual tautomers. For example the following structure:

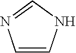

is understood to represent a mixture of the structures:

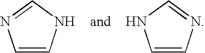

The term “pharmaceutically-acceptable salt” refers to salts which retain the biological effectiveness and properties of the compounds of the present invention and which are not biologically or otherwise undesirable. In many cases, the compounds of the present invention are capable of forming acid and/or base salts by virtue of the presence of amino and/or carboxyl groups or groups similar thereto.

### Embodiments

The present invention further encompasses use of the yeast two-hybrid system to identify regulators of the proteins and pathways described herein. Such regulators can be drugs, compounds, peptides, nucleic acids, etc. Such regulators can include endogenous regulators.

Generally, the yeast two-hybrid assay can identify novel protein-protein interactions and compounds that alter those interactions. By using a number of different proteins as potential binding partners, it is possible to detect interactions that were previously uncharacterized. Secondly, the yeast two-hybrid assay can be used to characterize interactions already known to occur. Characterization could include determining which protein domains are responsible for the interaction, by using truncated proteins, or under what conditions interactions take place, by altering the intracellular environment. These assays can also be used to screen modulators of the interactions.

This invention encompasses methods of screening compounds to identify those compounds that act as agonists (stimulate) or antagonists (inhibit) of the protein interactions and pathways described herein. Screening assays for antagonist compound candidates are designed to identify compounds that bind or complex with the peptides described herein, or otherwise interfere with the interaction of the peptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the compound or drug candidate with a peptide identified herein under conditions and for a time sufficient to allow these two components to interact.

In binding assays, the interaction is binding and the complex formed can be isolated or detected in the reaction mixture. In a particular embodiment, one of the peptides of the complexes described herein, or the test compound or drug candidate is immobilized on a solid phase, e.g., on a microtiter plate, by covalent or non-covalent attachments. Non-covalent attachment generally is accomplished by coating the solid surface with a solution of the peptide and drying. Alternatively, an immobilized antibody, e.g., a monoclonal antibody, specific for the peptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with, but does not bind to a particular peptide identified herein, its interaction with that peptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Complete kits for identifying protein-protein interactions between two specific proteins using the two-hybrid technique are available. This system can also be extended to map protein domains involved in specific protein interactions as well as to pinpoint amino acid residues that are crucial for these interactions.

Compounds that interfere with the interaction of a peptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the peptide may be added to a cell along with the compound to be screened for a particular activity and the ability of the compound to inhibit the activity of interest in the presence of the peptide indicates that the compound is an antagonist to the peptide. The peptide can be labeled, such as by radioactivity.

Other assays and libraries are encompassed within the invention, such as the use of Phylomers® and reverse yeast two-hybrid assays (see Watt, 2006, Nature Biotechnology, 24:177; Watt, U.S. Pat. No. 6,994,982; Watt, U.S. Pat. Pub. No. 2005/0287580; Watt, U.S. Pat. No. 6,510,495; Barr et al., 2004, J. Biol. Chem., 279:41:43178-43189; the contents of each of these publications is hereby incorporated by reference herein in their entirety). Phylomers® are derived from sub domains of natural proteins, which makes them potentially more stable than conventional short random peptides. Phylomers® are sourced from biological genomes that are not human in origin. This feature significantly enhances the potency associated with Phylomers® against human protein targets. Phylogica's current Phylomer® library has a complexity of 50 million clones, which is comparable with the numerical complexity of random peptide or antibody Fab fragment libraries. An Interacting Peptide Library, consisting of 63 million peptides fused to the B42 activation domain, can be used to isolate peptides capable of binding to a target protein in a forward yeast two hybrid screen. The second is a Blocking Peptide Library made up of over 2 million peptides that can be used to screen for peptides capable of disrupting a specific protein interaction using the reverse two-hybrid system.

The Phylomer® library consists of protein fragments, which have been sourced from a diverse range of bacterial genomes. The libraries are highly enriched for stable subdomains (15-50 amino acids long). This technology can be integrated with high throughput screening techniques such as phage display and reverse yeast two-hybrid traps.

Antibodies directed against proteins, polypeptides, or peptide fragments thereof of the invention may be generated using methods that are well known in the art. For instance, U.S. patent application Ser. No. 07/481,491, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For the preparation of monoclonal antibodies, any technique which provides for the production of antibody molecules by continuous cell lines in culture may be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) may be employed to produce human monoclonal antibodies. In another embodiment, monoclonal antibodies are produced in germ-free animals utilizing the technology described in international application no. PCT/US90/02545, which is incorporated by reference herein in its entirety.

In accordance with the invention, human antibodies may be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be employed; such antibodies are within the scope of the present invention. Once specific monoclonal antibodies have been developed, the preparation of mutants and variants thereof by conventional techniques is also available.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the invention.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12 (3,4): 125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters. Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for homologs of proteins and peptides. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function.

Conservative amino acid substitutions typically include substitutions within the following groups:

glycine, alanine;
valine, isoleucine, leucine;
aspartic acid, glutamic acid;
asparagine, glutamine;
serine, threonine;
lysine, arginine;
phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The present invention also provides nucleic acids encoding peptides, proteins, and antibodies of the invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89 (polylysine condensation of DNA in the form of adenovirus).

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2—S—CH2), dimethylene-sulfoxide (—CH2—SO—CH2), dimethylene-sulfone (—CH2—SO2—CH2), 2′—O— alkyl, and 2′-deoxy2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

The nucleic acids may be purified by any suitable means, as are well known in the art. For example, the nucleic: acids can be purified by reverse phase or ion exchange HPLC, size exclusion chromatography or gel electrophoresis. Of course, the skilled artisan will recognize that the method of purification will depend in part on the size of the DNA to be purified.

The term nucleic acid also specifically includes nucleic acids composed of bases other than the five biologically occurring bases (adenine, guanine, thymine, cytosine and uracil).

The present invention is directed to useful aptamers. In one embodiment, an aptamer is a compound that is selected in vitro to bind preferentially to another compound (in this case the identified proteins). In one aspect, aptamers are nucleic acids or peptides, because random sequences can be readily generated from nucleotides or amino acids (both naturally occurring or synthetically made) in large numbers but of course they need not be limited to these. In another aspect, the nucleic acid aptamers are short strands of DNA that bind protein targets. In one aspect, the aptamers are oligonucleotide aptamers. Oligonucleotide aptamers are oligonucleotides which can bind to a specific protein sequence of interest. A general method of identifying aptamers is to start with partially degenerate oligonucleotides, and then simultaneously screen the many thousands of oligonucleotides for the ability to bind to a desired protein. The bound oligonucleotide can be eluted from the protein and sequenced to identify the specific recognition sequence. Transfer of large amounts of a chemically stabilized aptamer into cells can result in specific binding to a polypeptide of interest, thereby blocking its function. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

As used herein, an antagonist or blocking agent may comprise, without limitation, an antibody, an antigen binding portion thereof or a biosynthetic antibody binding site that binds a particular target protein; an antisense molecule that hybridizes in vivo to a nucleic acid encoding a target protein or a regulatory element associated therewith, or a ribozyme, aptamer, or small molecule that binds to and/or inhibits a target protein, or that binds to and/or inhibits, reduces or otherwise modulates expression of nucleic acid encoding a target protein.

Aptamers offer advantages over other oligonucleotide-based approaches that artificially interfere with target gene function due to their ability to bind protein products of these genes with high affinity and specificity. However, RNA aptamers can be limited in their ability to target intracellular proteins since even nuclease-resistant aptamers do not efficiently enter the intracellular compartments. Moreover, attempts at expressing RNA aptamers within mammalian cells through vector-based approaches have been hampered by the presence of additional flanking sequences in expressed RNA aptamers, which may alter their functional conformation.

The idea of using single-stranded nucleic acids (DNA and RNA aptamers) to target protein molecules is based on the ability of short sequences (20 mers to 80 mers) to fold into unique 3D conformations that enable them to bind targeted proteins with high affinity and specificity. RNA aptamers have been expressed successfully inside eukaryotic cells, such as yeast and multicellular organisms, and have been shown to have inhibitory effects on their targeted proteins in the cellular environment.

The present application discloses compositions and methods for inhibiting the proteins described herein, and those not disclosed which are known in the art are encompassed within the invention. For example, various modulators/effectors are known, e.g. antibodies, biologically active nucleic acids, such as antisense molecules, RNAi molecules, or ribozymes, aptamers, peptides or low-molecular weight organic compounds recognizing said polynucleotides or polypeptides.

The present invention is also directed to pharmaceutical compositions comprising the vascular permeability regulatory compounds of the present invention. More particularly, such compounds can be formulated as pharmaceutical compositions using standard pharmaceutically acceptable carriers, fillers, solublizing agents and stabilizers known to those skilled in the art.

Pharmaceutically-acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. The generation of polyclonal antibodies is accomplished by inoculating the desired animal with the antigen and isolating antibodies which specifically bind the antigen therefrom.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89 (polylysine condensation of DNA in the form of adenovirus).

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2—S—CH2), dimethylene-sulfoxide (—CH2—SO—CH2), dimethylene-sulfone (—CH2—SO2-CH2), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

The nucleic acids may be purified by any suitable means, as are well known in the art. For example, the nucleic: acids can be purified by reverse phase or ion exchange HPLC, size exclusion chromatography or gel electrophoresis. Of course, the skilled artisan will recognize that the method of purification will depend in part on the size of the DNA to be purified.

The term nucleic acid also specifically includes nucleic acids composed of bases other than the five biologically occurring bases (adenine, guanine, thymine, cytosine and uracil).

Modified gene sequences, i.e. genes having sequences that differ from the gene sequences encoding the naturally-occurring proteins, are also encompassed by the invention, so long as the modified gene still encodes a protein that functions to stimulate healing in any direct or indirect manner. These modified gene sequences include modifications caused by point mutations, modifications due to the degeneracy of the genetic code or naturally occurring allelic variants, and further modifications that have been introduced by genetic engineering, i.e., by the hand of man. Techniques for introducing changes in nucleotide sequences that are designed to alter the functional properties of the encoded proteins or polypeptides are well known in the art. Such modifications include the deletion, insertion, or substitution of bases, and thus, changes in the amino acid sequence. Changes may be made to increase the activity of a protein, to increase its biological stability or half-life, to change its glycosylation pattern, and the like. All such modifications to the nucleotide sequences encoding such proteins are encompassed by this invention.

Oligonucleotides which contain at least one phosphorothioate modification are known to confer upon the oligonucleotide enhanced resistance to nucleases. Specific examples of modified oligonucleotides include those which contain phosphorothioate, phosphotriester, methyl phosphonate, short chain alkyl or cycloalkyl intersugar linkages, or short chain heteroatomic or heterocyclic intersugar (“backbone”) linkages. In addition, oligonucleotides having morpholino backbone structures (U.S. Pat. No. 5,034,506) or polyamide backbone structures (Nielsen et al., 1991, Science 254: 1497) may also be used.

The examples of oligonucleotide modifications described herein are not exhaustive and it is understood that the invention includes additional modifications of the antisense oligonucleotides of the invention which modifications serve to enhance the therapeutic properties of the antisense oligonucleotide without appreciable alteration of the basic sequence of the antisense oligonucleotide.

The invention also encompasses the use pharmaceutical compositions of an appropriate compound, homolog, fragment, analog, or derivative thereof to practice the methods of the invention, the composition comprising at least one appropriate compound, homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day. Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

Compounds which are identified using any of the methods described herein may be formulated and administered to a mammal for treatment of the diseases disclosed herein are now described.

The invention encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology. A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin. Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parenterally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self propelling solvent/powder dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1, to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein. A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1/1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the subject.

The compound may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the subject, etc.

The invention also includes a kit comprising a compound of the invention and an instructional material which describes administering the composition to a cell or a tissue of a subject. In another embodiment, this kit comprises a (preferably sterile) solvent suitable for dissolving or suspending the composition of the invention prior to administering the compound to the subject. The invention also provides a kit for identifying a regulator of vascular permeability as described herein, said kit comprising a sample of tissue or cells comprising tau and tubulin, Aβ, an applicator, and an instructional material for the use thereof.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

The invention is now described with reference to the following Examples and Embodiments. Without further description, it is believed that one of ordinary skill in the art can, using the preceding description and the following illustrative examples, make and utilize the present invention and practice the claimed methods. The following working examples therefore, are provided for the purpose of illustration only and specifically point out some embodiments of the present invention, and are not to be construed as limiting in any way the remainder of the disclosure. Therefore, the examples should be construed to encompass any and all variations which become evident as a result of the teaching provided herein.

### Example 1

### Materials and Methods

Antibodies: Tau-5, Tau-1, and R1 tau were generous gifts of Dr. Lester Binder, and PHF-1 was kindly provided by Dr. Peter Davies. AT180 (Pierce Endogen), DM1α (Sigma Chemical), Actin C4 (Chemicon), fluorescently tagged goat anti-mouse IgG and goat anti-rabbit (Southern Biotech), and HRP-labeled goat anti-mouse IgG (KPL) were acquired from the indicated commercial sources.

Cell Culture: CV-1 (African green monkey kidney) cells were cultured in DMEM (Dulbecco's modified Eagle's Medium; GIBCO) supplemented with 10% Cosmic Calf Serum (Hyclone) and 50 μg/ml gentamycin. Cells were transiently transfected using Fugene (Roche) with cDNAs for the longest human isoform of tau, or the projection domain (amino acids 1-248) of tau linked at their C-terminals to eCFP or eYFP (Clontech); with YFP-α-tubulin (Clontech); or with GFP-MAP2c or GFP-MAP2c/tau chimeras (Roger et al., 2004), which were generous gifts of Dr. Shelley Halpain. Primary cortical neurons were purchased from Genlantis and cultured according to their guidelines. Primary hippocampal neurons (Wisco et al., 2003) were grown for at least 8 days prior to Aβ treatment. The tau siRNA (Darmacon SMARTpool) and control scrambled siRNA (Darmacon non-specific duplex II) were transfected into primary hippocampal neurons using the rat neuron Nucleofection system (Amaxa) and program G-13 (Qiang et al., 2006). Cells were cultured for 4 days after Nucleofection, and then were exposed to Aβ.

Aβ treatment: Previously described methods were used to synthesize (Burdick et al., 1992) and resuspend (Kayed et al., 2003) Aβ42 and Aβ40. The Aβ was added to cells cultured in serum-free DMEM to final concentrations from 0.1-5 μM. Pre-fibrillar Aβ was used in the first and second days following resuspension, while fibrillar Aβ was used following at least 7 days of stirring.

Microscopy: Confocal live cell imaging and immunofluorescence microscopy were performed on a Zeiss Axiovert 100 using Openlab 4.0 (Improvision) software, as previously described (Mateer et al., 2002). For electron microscopy, primary cortical and hippocampal neurons were grown on glass coverslips, treated with Aβ42, and fixed in 2.5% glutaraldehyde+0.5% tannic acid in 0.1 M cacodylate buffer pH 7.4. Cells on coverslips were dehydrated and capsule embedded in EPON, and the glass coverslip was removed from the EPON by alternating liquid nitrogen and warm water submersion of the capsule. Sectioned samples were viewed on a JEOL JEM 1010 electron microscope at 80 kV and images captured using a SA1-12c 16 megapixel cooled CCD (Scientific Instruments and Applications, Inc).

Microtubule Extraction/Blotting: Primary hippocampal neurons were treated with 1-3 μM pre-fibrillar or fibrillar Aβ42. Cells were washed with PBS, and extracted with Triton X-100 in a microtubule stabilizing buffer (Black et al., 1996). Briefly, cells were incubated with PHEM buffer (60 mM PIPES pH 6.9, 25 mM HEPES, 10 mM EGTA, 2 mM MgCl₂) with 10 μM taxol, and 0.2% Triton X-100 for 5 minutes. The buffer was collected and centrifuged for 5 minutes at maximum speed in an Eppendorf model 5415 table top centrifuge, and the supernatant removed and added to sample buffer. An equivalent volume of PHEM buffer and sample buffer was added to the dish, and this sample was added to the pellet from the spin. Equal volumes of supernatants and pellets, which respectively contained soluble and polymerized tubulin (Black et al., 1996), were then analyzed by immunoblotting with DM1α monoclonal anti-α-tubulin (Sigma) and monoclonal tau-5. DM1α (Blose et al., 1984) and tau-5 (Loomis et al., 1990) recognize all isoforms or α-tubulin and tau, respectively, independently of post-translational modifications. Quantitation of scanned immunoreactive α-tubulin bands was performed using the public domain image processing software, Image J at the NIH website.

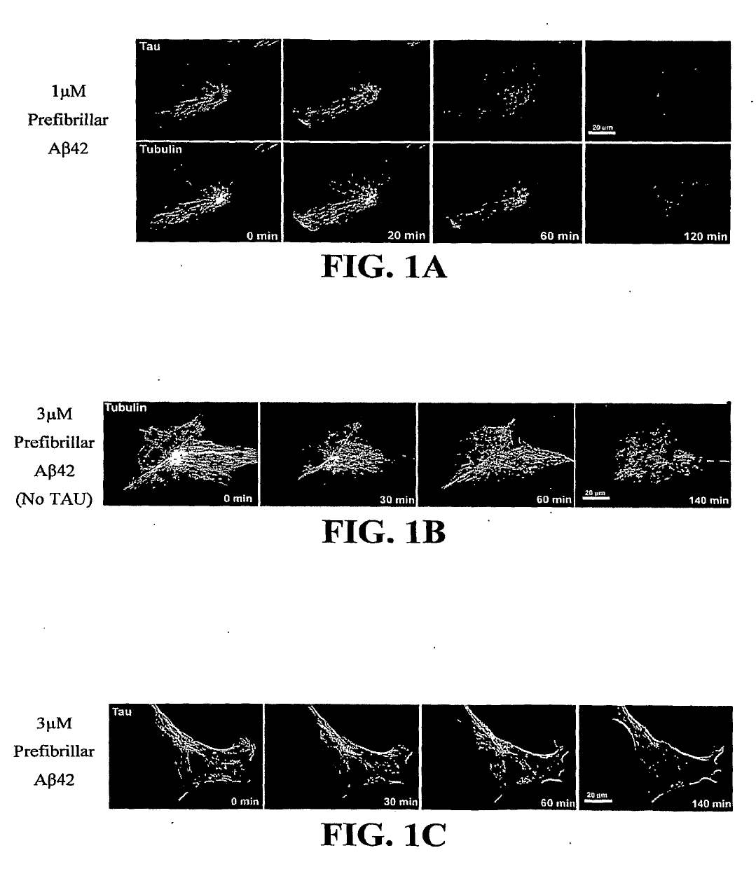

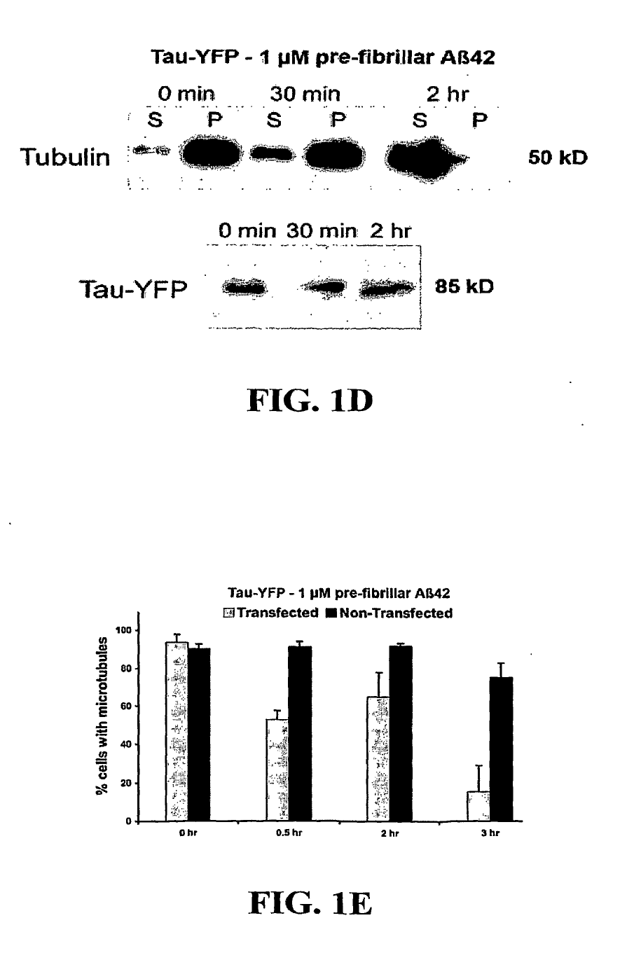

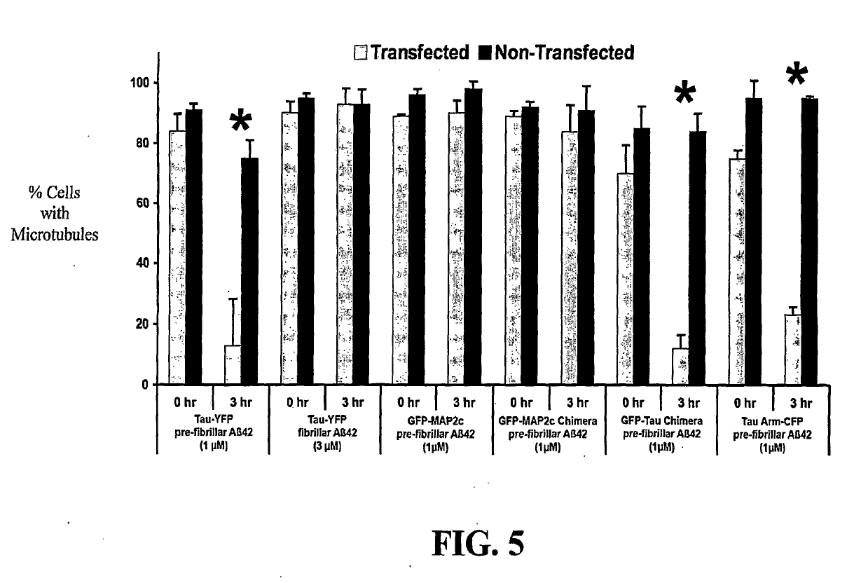

Quantitation of fluorescence micrographs: Nucleofection was used to express fusion proteins of CFP, GFP, or YFP coupled to tau, the N-terminal tau arm, MAP2c, or MAP2c/tau chimeras in CV-1 cells growing on glass coverslips. Cultures that either were or were not exposed to prefibrillar or fibrillar Aβ42 for 3 h were fixed and permeabilized for 5 min in −20° C. methanol and stained for immunofluorescence with anti-tubulin followed by TRITC-labeled goat anti-mouse IgG. For each coverslip, six randomly chosen fields of view were photographed separately in both the TRITC channel and the CFP, GFP, or YFP channel using the 25× objective. Typically, 40-50% of the total cells expressed the transfected protein. Next, without knowing the identity of the sample, an observer counted the total cells and microtubule-containing cells in one anti-tubulin field and then counted the total number of transfected cells and microtubule-containing transfected cells in the same field. This process was repeated for the remaining fields of the coverslip, and the results from all six fields, which comprised 500 total cells, were merged into a single dataset. Each such experiment was performed in triplicate, and the net results were graphed in FIGS. 1E and 5 as the mean±SD of the percentage of microtubule-containing transfected and nontransfected cells for each experimental condition. For FIG. 5, pairwise comparisons were made of transfected versus nontransfected cells at 0 and 3 hours of Aβ exposure and of nontransfected cells at 0 versus 3 hours of Aβ exposure.

### Results and Discussion

Co-expression of tau-CFP and YFP-tubulin in CV-1 African green monkey kidney cells, which do not express endogenous tau, allowed effects of various forms of Aβ on tau and tubulin distributions to be monitored in live cells by time lapse fluorescence microscopy. Aβ is known to transition gradually from monomers to oligomers, protofibrils, and finally to highly stable fibrils (Bitan et al., 2003). Because no consistent differences in behavior were observed between freshly solubilized Aβ, which contains a mixture of monomers, dimers and tetramers, versus Aβ recognized by an antibody that detects oligomers containing a minimum of eight Aβ subunits (Kayed et al., 2003), these forms of Aβ are collectively referred to as “pre-fibrillar”. Within 1-2 hours after adding 0.1-3 μM pre-fibrillar Aβ42 to culture media, tau dissociated from microtubules, which completely disassembled soon thereafter (FIG. 1a). Pre-fibrillar Aβ40 was also capable of inducing tau-dependent microtubule disassembly in CV-1 cells, but at a minimum concentration of 3 μM. In contrast, microtubules remained intact for more than two hours when CV-1 cells expressing YFP-tubulin, but not tau-CFP, were exposed to as much as 3 μM pre-fibrillar Aβ42 (FIG. 1b). Similarly, microtubule integrity was unaffected in cells expressing tau-CFP after more than two hours of exposure to as much as 5 μM fibrillar Aβ42 (FIG. 1c). We thus conclude that tau makes CV-1 cell microtubules hypersensitive to pre-fibrillar Aβ peptides, particularly Aβ42, but not to fibrillar Aβ42.

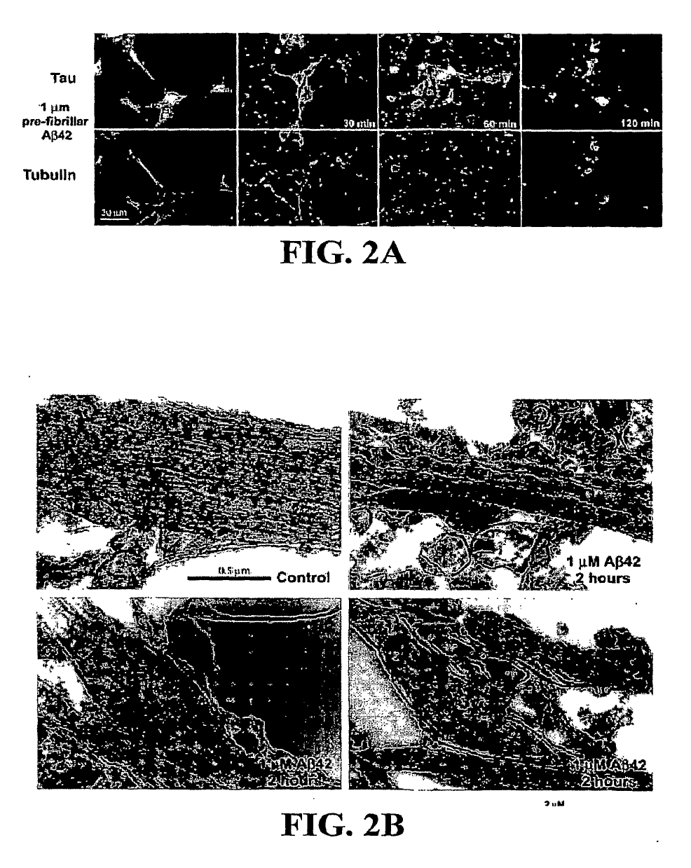

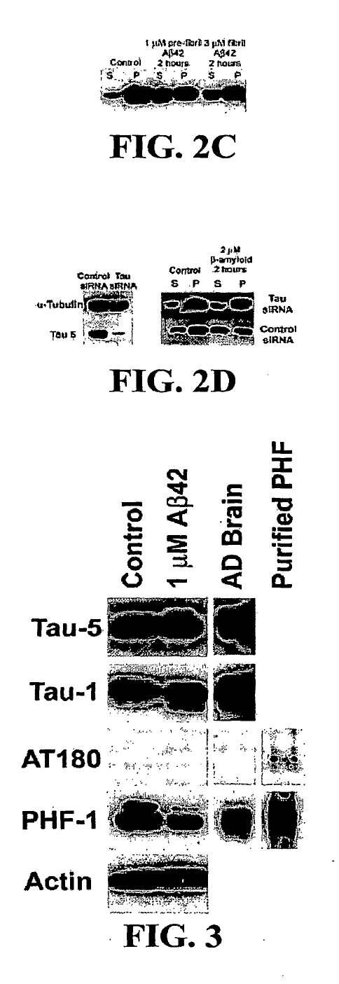

Microtubule disassembly was also observed in primary rat cortical neurons following exposure to pre-fibrillar Aβ42. Discrimination between polymerized and disassembled tubulin was not evident from immunostaining of the treated neurons. Nevertheless, the immunofluorescence images show that prior to pre-fibrillar Aβ42 addition, the cells displayed numerous neuritic projections (FIG. 2a). Following only 30 minutes of cellular exposure to 1 μM pre-fibrillar Aβ42, the neurites displayed swollen varicosities, and at later time points the cells appeared to lose the majority of their neuritic projections. Similar cultures were examined by electron microscopy (FIG. 2b). Neurites of control cells typically contained densely packed microtubules arranged in parallel. In contrast, neurites in cells treated with pre-fibrillar Aβ42 contained fewer, less organized microtubules, and conspicuous swellings that were filled with membrane-bound organelles and were virtually devoid of microtubules. Similar findings have been reported for primary cortical neurons exposed to 5 μM oligomeric Aβ40 for 3-6 hours (Fifre et al., 2006; Sponne et al., 2003).

A well established biochemical assay that partitions un-polymerized and polymerized tubulin into Triton X-100 soluble and insoluble fractions, respectively (Black et al., 1996), was used to monitor effects of pre-fibrillar and fibrillar Aβ42 on microtubule integrity (FIG. 2c). About 90% of the tubulin was polymerized in cells that were not exposed to Aβ42, but only 45% of the tubulin was polymerized after 2 hours of cellular exposure to 1 μM pre-fibrillar Aβ42. Some microtubule loss (65% polymerized) was observed after a comparable exposure to fibrillar Aβ42, but only at the much higher total peptide concentration of 3 μM.

Tau was found to be required for microtubule disassembly in primary hippocampal neurons induced by pre-fibrillar Aβ42. Neurons were treated with siRNA to reduce tau expression to trace levels (FIG. 2d), and following 2 hours of exposure to 2 μM pre-fibrillar Aβ 42, the level of polymerized tubulin remained nearly unchanged in tau-deficient neurons (˜20% soluble tubulin), while the tau expressing neurons again showed an increase in soluble tubulin (˜60% soluble tubulin). It was not possible to determine how much tau was microtubule-bound or soluble in these experiments, because the tau was quantitatively solubilized by the Triton X-100 under conditions in which the polymerized tubulin was resistant to extraction (Black et al., 1996). Thus, the results for tubulin demonstrate that the endogenous tau in neurons, like transfected tau in CV-1 cells, makes microtubules acutely sensitive to pre-fibrillar Aβ42.

Treatment of tau-expressing neurons with pre-fibrillar Aβ42 under conditions that induced microtubule disassembly did not cause increased AD-like tau phosphorylation at any of several sites (FIG. 3). This was found by immunoblotting using phosphorylation-sensitive monoclonal anti-tau antibodies: PHF-1, AT180, and tau-1. Although many additional AD-like phosphorylation sites remain to be examined, these data suggest that conversion of tau to an AD-like phosphorylation state does not underlie the release of tau from microtubules and subsequent microtubule disassembly induced by pre-fibrillar Aβ42.

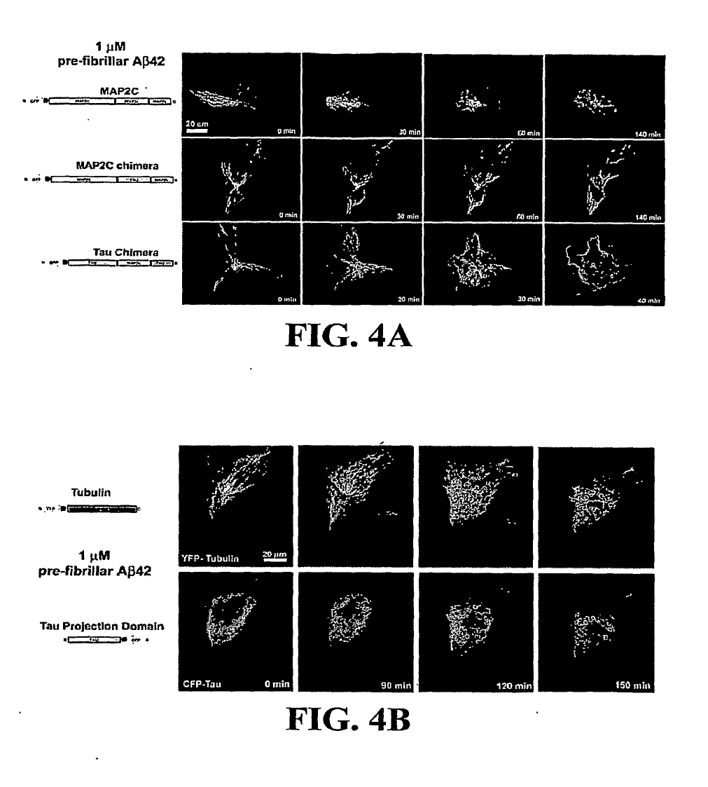

The specificity of tau for microtubule loss in cells treated with pre-fibrillar Aβ42 was shown by expressing GFP-tagged MAP2c in CV-1 cells. MAP2c is a neuron-specific microtubule-associated protein with a microtubule binding domain ˜-70% identical to that of tau's (Dehmelt and Halpain, 2005). Over 2 hours of exposure to pre-fibrillar Aβ42 did not cause any apparent loss of microtubule integrity in cells. A region of tau responsible for conferring sensitivity to Aβ was mapped using a combination of tau/MAP2c chimeric proteins and a CFP-tagged tau fragment. Only cells expressing a GFP-tagged chimera of the microtubule binding domain of MAP2c flanked by the N-terminal arm and C-terminal tail of tau (FIG. 4a) responded to the addition of pre-fibrillar Aβ42. Similar activity was observed when the tau projection domain-CFP, which did not localize on microtubules, was expressed in cells that subsequently were treated with pre-fibrillar Aβ42 (FIG. 4b). The N-terminal half of tau therefore responds to pre-fibrillar Aβ42, and does not have to target to microtubules to exert its effects. Furthermore, the closely related neuronal microtubule protein, MAP2c, cannot substitute for tau at promoting microtubule disassembly in cells exposed to pre-fibrillar Aβ42.

The nature of the functional connection between Aβ and tau has been one of the most enduring and intractable mysteries in AD research, and solving this mystery is bound to open potential new avenues of early detection and therapeutic intervention for AD. The results presented here represent the swiftest and most deleterious tau-dependent effects of Aβ that have yet been described. When considered together with recently published studies demonstrating localization of oligomeric Aβ in AD brain at sites distinct from classic amyloid plaques (Kayed et al., 2003), a critical role for oligomeric Aβ in memory loss (Lesne et al., 2006), and co-localization of Aβ with tau in tangle-bearing AD neurons in vivo (Guo et al., 2006), the present results suggest a mechanism by which Aβ and tau conspire coordinately to compromise neuronal function. Diminished microtubule integrity could compromise essential microtubule functions, such as fast axonal transport, which is required to maintain synapses, and delivery of exocytotic membranes to the cell surface to repair plasma membrane holes (Shen et al., 2005) known to be induced by oligomeric Aβ (Kayed et al., 2003). That the combination of pre-fibrillar Aβ and non-filamentous tau were able to elicit such a dramatic disruption of microtubules supports the hypothesis that fibrillar forms of tau and Aβ are at least somewhat neuro-protective, because they sequester more dangerous, non-fibrillar forms of Aβ and tau (Tanzi, 2005). The fact that tau is required for Aβ-induced microtubule loss could explain, at least in part, why neurons, the principal tau-expressing cell type, are the cellular targets for destruction in AD. Moreover, the model presented here does not preclude other toxic functions of pre-fibrillar or fibrillar Aβ or filamentous tau, such as tau-dependent degeneration of cultured neurons induced by fibrillar Aβ40 (Rapoport et al., 2002) or toxicity related to intracellular tau filament accumulation (Khlistunova et al., 2006). Nevertheless, the rapid, tau-dependent destruction of microtubules that we observed to be induced by submicromolar concentrations of pre-fibrillar Aβ42 suggests this process is one of the seminal events in AD pathogenesis at the cellular level.

### BIBLIOGRAPHY

Bitan et al., 2003, Proc. Natl. Acad. Sci. U.S.A. 100:330-5.
Black, M. M., T. Slaughter, S. Moshiach, M. Obrocka, and I. Fischer. 1996. Tau is enriched on dynamic microtubules in the distal region of growing axons. J. Neurosci. 16:3601-19.
Blose, S. H., D. I. Meltzer, and J. R. Feramisco. 1984. 10-nm filaments are induced to collapse in living cells microinjected with monoclonal and polyclonal antibodies against tubulin. J. Cell Biol. 98:847-58.
Burdick, D., B. Soreghan, M. Kwon, J. Kosmoski, M. Knauer, A. Henschen, J. Yates, C. Cotman, and C. Glabe. 1992. Assembly and aggregation properties of synthetic Alzheimer's A4/beta amyloid peptide analogs. J Biol. Chem. 267:546-54.
Dehmelt, L., and S. Halpain. 2005. The MAP2/Tau family of microtubule-associated proteins. Genome Biol. 6:204.
Fifre, A., I. Sponne, V. Koziel, B. Kriem, F. T. Yen Potin, B. E. Bihain, J. L. Olivier, T. Oster, and T. Pillot. 2006. Microtubule-associated protein MAP1A, MAP1 B, and MAP2 proteolysis during soluble amyloid beta-peptide-induced neuronal apoptosis. Synergistic involvement of calpain and caspase-3. J Biol. Chem. 281:229-40.
Gotz, J., F. Chen, J. van Dorpe, and R. M. Nitsch. 2001. Formation of neurofibrillary tangles in P3011 tau transgenic mice induced by Abeta 42 fibrils. Science. 293:1491-5.
Guo, J. P., T. Arai, J. Miklossy, and P. L. McGeer. 2006. Abeta and tau form soluble complexes that may promote self aggregation of both into the insoluble forms observed in Alzheimer's disease. Proc Natl Acad Sci USA. 103:1953-8.
Hardy, J., and D. J. Selkoe. 2002. The amyloid hypothesis of Alzheimer's disease: progress and problems on the road to therapeutics. Science. 297:353-6.
Kayed, R., E. Head, J. L. Thompson, T. M. McIntire, S. C. Milton, C. W. Cotman, and C. G. Glabe. 2003. Common structure of soluble amyloid oligomers implies common mechanism of pathogenesis. Science. 300:486-9.
Khlistunova, I., J. Biernat, Y. Wang, M. Pickhardt, M. von Bergen, Z. Gazova, E. Mandelkow, and E. M. Mandelkow. 2006. Inducible expression of Tau repeat domain in cell models of tauopathy: aggregation is toxic to cells but can be reversed by inhibitor drugs. J Biol Chem. 281:1205-14.
Lee, V. M., M. Goedert, and J. Q. Trojanowski. 2001. Neurodegenerative tauopathies. Annu Rev Neurosci. 24:1121-59.
Lesne, S., M. T. Koh, L. Kotilinek, R. Kayed, C. G. Glabe, A. Yang, M. Gallagher, and K. H. Ashe. 2006. A specific amyloid-beta protein assembly in the brain impairs memory. Nature. 440:352-7.
Lewis, J., D. W. Dickson, W. L. Lin, L. Chisholm, A. Corral, G. Jones, S. H. Yen, N. Sahara, L. Skipper, D. Yager, C. Eckman, J. Hardy, M. Hutton, and E. McGowan. 2001. Enhanced neurofibrillary degeneration in transgenic mice expressing mutant tau and APP. Science. 293:1487-91.
Loomis, P. A., T. H. Howard, R. P. Castleberry, and L. I. Binder. 1990. Identification of nuclear tau isoforms in human neuroblastoma cells. Proc Natl Acad Sci USA. 87:8422-6.
Mateer, S. C., A. E. McDaniel, V. Nicolas, G. M. Habermacher, M.-J. S. Lin, D. A. Cromer, M. E. King, and G. S. Bloom. 2002. The Mechanism for Regulation of the F-actin Binding Activity of IQGAP1 by Calcium/Calmodulin. J. Biol. Chem. 277:12324-12333.
Novak, M., J. Kabat, and C. M. Wischik. 1993. Molecular characterization of the minimal protease resistant tau unit of the Alzheimer's disease paired helical filament. Embo J. 12:365-70.
Oddo, S., L. Billings, J. P. Kesslak, D. H. Cribbs, and F. M. LaFerla. 2004. Abeta immunotherapy leads to clearance of early, but not late, hyperphosphorylated tau aggregates via the proteasome. Neuron. 43:321-32.
Oddo, S., A. Caccamo, L. Tran, M. P. Lambert, C. G. Glabe, W. L. Klein, and F. M. Laferla. 2005. Temporal profile of Abeta oligomerization in an in vivo model of Alzheimer's disease: A link between Abeta and tau pathology. J Biol. Chem.
Qiang, L., W. Yu, A. Andreadis, M. Luo, and P. W. Baas. 2006. Tau protects microtubules in the axon from severing by katanin. J. Neurosci. 26:3120-9.
Rapoport, M., H. N. Dawson, L. I. Binder, M. P. Vitek, and A. Ferreira. 2002. Tau is essential to beta-amyloid-induced neurotoxicity. Proc Natl Acad Sci USA. 99:6364-9.
Roger, B., J. AI-Bassam, L. Dehmelt, R. A. Milligan, and S. Halpain. 2004. MAP2c, but not tau, binds and bundles F-actin via its microtubule binding domain. Curr Biol. 14:363-71.
Selkoe, D. J. 2001. Alzheimer's disease: genes, proteins, and therapy. Physiol Rev. 81:741-66.
Shen, S. S., W. C. Tucker, E. R. Chapman, and R. A. Steinhardt. 2005. Molecular regulation of membrane resealing in 3T3 fibroblasts. J Biol. Chem. 280:1652-60.
Sponne, I., A. Fifre, B. Drouet, C. Klein, V. Koziel, M. Pincon-Raymond, J. L. Olivier, J. Chambaz, and T. Pillot. 2003. Apoptotic neuronal cell death induced by the non-fibrillar amyloid-beta peptide proceeds through an early reactive oxygen species-dependent cytoskeleton perturbation. J Biol. Chem. 278:3437-45.
Tanzi, R. E. 2005. Tangles and neurodegenerative disease—a surprising twist. N Engl J. Med. 353:1853-5.
Walsh, D. M., I. Klyubin, J. V. Fadeeva, W. K. Cullen, R. Anwyl, M. S. Wolfe, M. J. Rowan, and D. J. Selkoe. 2002. Naturally secreted oligomers of amyloid beta protein potently inhibit hippocampal long-term potentiation in vivo. Nature. 416:535-9.
Wisco, D., E. D. Anderson, M. C. Chang, C. Norden, T. Boiko, H. Folsch, and B. Winckler. 2003. Uncovering multiple axonal targeting pathways in hippocampal neurons. J. Cell Biol. 162:1317-28.

The disclosures of each and every patent, patent application, and publication cited herein are hereby incorporated by reference herein in their entirety. One of skill in the art will appreciate that the superiority of the compositions and methods of the invention relative to the compositions and methods of the prior art are unrelated to the physiological accuracy of the theory explaining the superior results.

Headings are included herein for reference and to aid in locating certain sections. These headings are not intended to limit the scope of the concepts described therein under, and these concepts may have applicability in other sections throughout the entire specification.

Other methods which were used but not described herein are well known and within the competence of one of ordinary skill in the art of clinical, chemical, cellular, histochemical, biochemical, molecular biology, microbiology and recombinant DNA techniques.

The description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

What is claimed is:

1. A method of identifying a compound that inhibits Aβ or tau mediated disassembly of microtubules, said method comprising:
a. culturing cells comprising tau and tubulin;
b. contacting said cells with at least one test compound;
c. contacting said cells with Aβ; and
d. analyzing the microtubules, wherein a higher level of microtubules in cells contacted with said at least one test compound, compared with the level of microtubules in otherwise identical cells not contacted with said at least one test compound, is an indication that said test compound inhibits Aβ or tau mediated disassembly of microtubules, thereby identifying a compound that inhibits Aβ or tau mediated disassembly of microtubules.

2. The method of claim 1, wherein said compound inhibits the interaction of the N-terminal domain of tau with said Aβ.

3. The method of claim 2, wherein said method identifies inhibitors of the interaction of the N-terminal domain of tau with said Aβ.

4. The method of claim 2, wherein said Aβ is pre-fibrillar Aβ.

5. The method of claim 1, wherein said compound regulates the interaction of tau with tubulin.

6. The method of claim 5, wherein a change in the interaction of tau with tubulin, or interaction of Aβ with tau, is associated with microtubule disassembly.

7. The method of claim 6, wherein said compound reduces the sensitivity of microtubule disassembly to tau mediated by Aβ.

8. The method of claim 6, wherein said compound inhibits the interaction of tau with Aβ.

9. The method of claim 1, wherein said Aβ is pre-fibrillar Aβ.

10. The method of claim 9, wherein said Aβ is Aβ40 or Aβ42.

11. The method of claim 1, wherein said cells are selected from the group consisting of primary neuronal cells, primary non-neuronal cells, cell lines, cells strains, and fibroblasts.

12. The method of claim 11, wherein said cells are fibroblasts.

13. The method of claim 11, wherein said primary neuronal cells are primary hippocampal cells.

14. The method of claim 1, wherein said cells are CV-1 African green monkey kidney cells.

15. The method of claim 1, wherein tau is not endogenously expressed in said cells.

16. The method of claim 1, wherein said tau and tubulin are labeled.

17. The method of claim 16, wherein said tau is fluorescent-labeled and said tubulin is fluorescent-labeled.

18. The method of claim 1, wherein said compound is identified using high throughput screening techniques.

19. The method of claim 18, wherein said compound is identified as part of a combinatorial library.

20. The method of claim 1, wherein said compound is selected from the group consisting of an interfering RNA, a small interfering RNA, an oligonucleotide, a protein, a peptide, an antibody, and an aptamer.

21. The method of claim 20, wherein said antibody is a monoclonal antibody.

22. The method of claim 20, wherein said small interfering RNA is directed against Aβ or tau.

23. The method of claim 1, wherein said compound is useful for treating Alzheimer's disease.

24. The method of claim 1, wherein said Aβ is added to the culture before said at least one test compound.

25. The method of claim 1, wherein said Aβ is added to the culture after said at least one test compound.

26. A compound identified by the method of claim 1.

27. An in vitro model for detecting and measuring early cellular events in Alzheimer's disease, said model comprising culturing cells in vitro, wherein said cells comprise tau and tubulin, contacting said cells with Aβ, and analyzing changes in microtubules.

28. The in vitro model of claim 27, wherein said cells do not endogenously express tau.

29. The in vitro model of claim 27, wherein tau and tubulin are labeled.

30. The in vitro model of claim 29, wherein said label is a fluorescent label.

31. The in vitro model of claim 27, wherein said cells are selected from the group consisting of primary neuronal cells, primary non-neuronal cells, cell lines, cells strains, and fibroblasts.

32. The in vitro model of claim 31, wherein said cells are CV-1 African green monkey kidney cells.

33. The in vitro model of claim 31, wherein said primary neuronal cells are primary hippocampal cells.

34. The in vitro model of claim 27, wherein said cells do not endogenously express Aβ.

35. A method of treating Alzheimer's disease in a subject in need thereof, said method comprising administering to said subject a pharmaceutical composition comprising an effective amount of at least one compound of claim 1 and a pharmaceutically-acceptable carrier, and optionally another Alzheimer's disease therapeutic agent.
